(19) United States
(12) Patent Application Publication
(10) Pub. No.: US 2021/0145621 A1
(43) Pub. Date: May 20, 2021

(54) Smart Custom Orthotic

(71) Applicant: Fiomet Ventures, Inc., Cincinnati, OH (US)

(72) Inventors: Scott J. Rapp, Cincinnati, OH (US); Christopher B. Gordon, Cincinnati, OH (US); Horacio L. Rilo, Cincinnati, OH (US); Jared Sartee, Pemberton (CA)

(21) Appl. No.: 17/159,242

(22) Filed: Jan. 27, 2021

Related U.S. Application Data
(63) Continuation-in-part of application No. 15/289,071, filed on Oct. 7, 2016.
(60) Provisional application No. 62/969,067, filed on Feb. 1, 2020.
(60) Provisional application No. 62/238,522, filed on Oct. 7, 2015.

(51) Int. Cl. A61F 5/058; A61B 5/02; A61B 5/00; A61B 5/145; A61B 5/01

(52) U.S. Cl. CPC A61F 5/05891; A61F 5/05816; A61B 5/02; A61B 5/6812; A61B 5/6803; A61B 2562/0271; A61B 5/14546; A61B 5/14542; A61B 5/14532; A61B 5/01; A61B 2562/063; A61B 5/14539

(57) ABSTRACT

A portable customizable smart helmet, cap or splint for wound care, trauma and critical care.

This Application claims the benefit of Provisional Application 62/969,067—Wound Treatment Devices—filed on Feb. 1, 2020 and priority to U.S. application Ser. No. 15/289,071—Advanced Compression Garments and Systems —, filed Oct. 7, 2016 that claimed the benefit of U.S. Provisional Application 62/238,522, filed on Oct. 7, 2015.

### BACKGROUND OF THE INVENTION

### A. Field of the Invention

The current smart custom orthotic is a portable smart helmet, cap or splint device utilized for custom orthotics, wound care, trauma and critical care. Features of the current invention can reduce post injury trauma and adverse physiological events while also providing notifications that can assist medical intervention. Among other things, the smart custom cranial orthotic can be adapted to customize pressure applied to the wound or abnormal environment.

It is estimated that over 13 million living people have symptoms related to traumatic brain injury in the US and Europe populations alone¹. Worldwide, the incidence is increasing due to the more readily available methods of motor transportation such as motorcycles and low-cost scooters. Even when helmeted, the transmitted force to the brain can lead to concussions, traumatic brain injury, to diffuse axonal injury (DAI) and death. It has been reported that over 20% of all concussions are sports-related in the US².

As such, multiple efforts are focused on improving the materials in both sports-related and commercial helmets for protection. The ability to disperse forces through the helmet structures may reduce the harmful impact of accelerations and decelerations on the brain movement within the calvarium. The geometric structure and substance of the padding portion of the helmet (i.e. prismatic lattice) have been shown to reduce TBI³.

Specific focus has been on custom liners for helmets such as viscoelastic polymers which have been shown to reduce strain and strain-rates for head impact¹. Penetrating brain injuries carry the highest mortality rates⁴. Retrospective analysis of the Iraq and Afghanistan wars revealed that numbers of penetrating TBIs exceeded closed TBIs by a ratio of 2:1 and 1.3:1 respectively⁵. The ability to control hemostasis with penetrating brain injuries in the field is quite difficult. Too much pressure can cause tonsillar herniation or brain injury and death, too little compression may result in excessive bleeding and shock. It is clear there is a need for specific wound care solutions in these scenarios.

In penetrating head trauma, closed head injuries and acquired cranial deformities from surgical intervention, multiple adverse clinical sequalae may result. The scalp provides a robust blood supply to the surrounding anatomic structures and disruption resulting from insult can lead to excessive blood loss and even death within a short period of time. Obtaining hemostasis is quite feasible in the operating theater or ambulatory medical setting, however in the field and away from medical help, scalp injuries may be difficult to control leading to hypotension, shock and death. Additionally, penetrating injuries to the scalp and underlying skull represent an even more severe clinical scenario where serious sequalae such as hemorrhagic shock, hypotension, hypothermia and low perfusion pressures can lead to further tissue damage and death. Lack of cerebral perfusion leads to hypoxemia and neuronal degradation. Hypotension and hypothermia in the face of a traumatic event have been shown to be critical contributing factors that lead to higher rates of secondary wound infections and significant downstream comorbidities.

Swelling from the above injuries can be rather profound at the time of injury, but may peak at 48 hours from injury. Compressive bandages are a first line treatment defense against hemorrhage. However, static application of the bandages without manipulation over the course of a physiologic response to injury may result in further injuries. Dressings applied at the initial time of injury may become too tight and in itself cause further injury or harm to the individual if variations to inflammation and swelling occur. Alternatively, hypobaric environments, such as during transport in flight also results in changes to both the perfusion pressures to the tissues as well as risk of wound infection.

Selective brain cooling has been shown provide neuroprotective capabilities after brain trauma. Induced hypothermia has been shown to minimize acute brain damage in animal models after penetrating ballistic-like brain injury (PBBI). These models have shown improved preservation of axonal integrity, cellular apoptosis, blood-brain barrier disruption, neuroinflammation and improved behavior outcomes both in TBI and PBBI. Reducing brain temperature by 2-3 degrees Celsius while maintaining corporal normothermia may have life-saving capabilities. Selectively cooling the brain may also allow for a greater time window to allow for transportation from the field to the medical facility where more effective strategies to treat repairable injuries may be obtained. However, focal cooling of the brain both in the field and in the medical arena without causing systemic lowering of temperature has been difficult to achieve in clinical practice. As above, hypothermia has been shown to be a critical factor in overall mortality, wound infection rates, blood coagulopathies, and overall hospital stay following severe traumatic injury.

### B. Description of the Previous Art

Any discussion of references cited herein merely summarizes the disclosures of the cited references. Applicant makes no admission that any cited reference or portion thereof is relevant prior art. Applicant reserves the right to challenge the accuracy, relevancy and veracity of the cited references. Publications or presentations that may indicate a state-of-art include:

1. Siegkas P, Sharp D J, Ghajari M. The traumatic brain injury mitigation effects of a new viscoelastic add-on liner. Sci Rep. 2019 Mar. 5; 9(1):3471.

2. National Center for Injury Prevention and Control. Report to Congress on mild traumatic brain injury in the United States: steps to prevent a serious public health problem. Atlanta, Ga.: Centers for Disease Control and Prevention, National Center for Injury Prevention and Control, 2003

3. Khosroshahi S F, Duckworth H, Galvanetto U, Ghajari M. The effects of topology and relative density of lattice liners on traumatic brain injury mitigation. J Biomech. 2019 Dec. 3; 97:109376.

4. Fathalla H, Ashry A, El-Fiki A. Managing military penetrating brain injuries in the war zone: lessons learned. Neurosurg Focus. 2018 Dec. 1; 45(6):E6.

5. Orman J A, Geyer D, Jones J, Schneider E B, Grafman J, Pugh M J, Dubose J. Epidemiology of moderate-to-severe penetrating versus closed traumatic brain injury in the Iraq and Afghanistan wars. J Trauma Acute Care Surg. 2012 December; 73(6 Suppl 5):S496-502.

### SUMMARY OF THE INVENTION

The present custom cranial orthotic device and system can address adverse events following trauma and can preserve brain hemodynamics, brain metabolism and neurobehavior during subacute injury period

The current device and system embodiments are directed towards a custom cranial orthotic that can provide real time biometric feedback from multiple different sensors. Therapeutic interventions such as hemostasis, compression, adaptive heating and cooling and improved wound care optimization are possible with the custom cranial orthotic device and system.

To address hemorrhage following injury, the orthotic has a custom foaming capability to adapt to any contour irregularity such as soft tissue scalp injury to even a concavity or convexity due to bony skull malposition. The foaming agents are able to expand into the defect or around the defect to provide focal compression and improved hemostasis. By providing diffuse pressure over a greater surface area in and around the wound, less pressure can be used to obtain hemostatic control. With penetrating injuries and concavities to the skull, the foam will expand into this area providing a more appropriate mold of the defect rather than conventional dressings that are difficult to contour in the face of a dirty or bleeding wound. A bladder interposition made of polyurethane or other polymeric materials may be implemented to ensure a watertight pressure application and enhanced rigidity to the foamed dressing.

Outside the foaming agents, a hard-collapsible shell can be applied. The shell is collapsible for ease of storage and transport on the individual or field equipment (i.e. ambulance or military vehicle). The composition of the shell will be strong enough that it can reduce against further traumatic events. The shell is also adjustable to provide finer and evenly distributed compression. Leaflets associated with the helmet provide control of external compression (more or less) supplied by the helmet or cap as required by medical parameters or when indicated by pressure sensors.

Within the scope of the current invention, biometric sensors can be positioned in the bladder, the outer shell or both. In select embodiments of the helmet or cap, biometric sensors such as pressure sensors can be place at multiple locations of the invention. Use of one or more pressure sensors can provide alerts indications and/or alerts of acceptable, excessive or insufficient pressures. Insufficient pressures may result in excessive hemorrhage, low cerebral perfusion pressures, secondary hypoxia and neurodegeneration such as axonal or glial death. Excessive pressures may result in brain herniation, soft tissue ischemia, cerebral vascular injury or stroke, and death. A biometric sensor communicates with communications module adapted to communicate with a computer distinct from the communications module. Communications module includes a transmitter or a transceiver allowing wireless communications with a remote computer that provides real real-time calculation/determination of the biometric(s) sensed. Communication modules can also include a memory, a microprocessor or processor and software (computer component) for calculating the biometric measurements sensed by the biometric sensors and controlling the helmet's heat/cooling semiconductors. Depending on medical treatment parameters, the remote computer or the communications module's computer module can be used to calculate sensed biometrics and control the heat/cooling semiconductors. In accordance with the present invention, the computer or computer module can calculate biometrics, including but not limited to, pressure, temperature, blood pressure, lactate levels, pH, sodium levels, potassium level, glucose levels, apoptotic factors, nitric oxide (NO) and SVO2 levels sensed by their respective sensors.

In selected embodiments of the current invention, semiconductors, such as micro-Peltier coolers, can be included in the bladder, the outer shell, foam or any combination to provide heating or cooling. The direction of the current flow through micro-semiconductors allows for either cooling or warming. Power for the semiconductors and other components of the current orthotic can be provided by batteries, a connection with an alternating current power source or a radio frequency energy supply.

Within the ambit of the current invention, micro-semiconductors can be rigid or flexible or a combination thereof. The semiconductors can utilize metallic thermal interfaces to dissipate heat or cold. Select preferred embodiments of the micro-semiconductors can utilize ferromagnetic foils that transition between paramagnetic and ferromagnetic states and can drive a metallic shuttle generating a squeezed-film cooling effect during oscillation. The thermal interfaces can induce vibration of fluids in proximity with the thermal interfaces. Select embodiments of the thermal interfaces can be provided with carbon nanotubes that can decrease thermal interface resistance. The carbon nanotubes can be grown on gadolinium foil. Use of carbon nanotubes can improve performance of the semiconductors.

It is believed that use of micro-semiconductors allows the application of different voltages and current across two distinct semiconductors, thereby generating a “heat flux” of hot or cold across the semiconductors' interfaces. Direction of current crossing the interfaces changes the generated heat flux from hot to cold or cold to hot such that the change the direction of current can result in a 25 degree Celsius deferential of temperature proximate the interfaces.

Helmet size and foam application can be of different dimensions. By way of illustration, caps sizes are available for the pediatric, adolescent and adult populations. Within the scope of the current invention, rather than a helmet, the outer shell can be oriented to fit around other body parts, limbs, torso or hands. Shells of the current invention can be provided with one or more tunnels that include one or fans. It is believed that assisting the circulation of cold or hot air at preselected temperatures can improve healing of the tissue. By way of illustration, induction of localized hypothermia can be utilized to protect tissue.

The current custom orthotic apparatus also has application in veterinary medicine. Custom foaming devices such as helmets and splints can be configured with external hard shells and adapted to treat and protect animal injuries from trauma or during the post-operative period for animals. Those animals include, but are not limited to equine, canine, feline or zoo animals.

An aspect of the present invention is to provide a customized orthotic.

Another aspect of the present invention is to provide an orthotic that is controlled by the orthotic's communications module or via wireless transmission from a computing device remote from the communications module.

Yet another aspect of the present invention is to provide an orthotic that provides adjustable pressures to different zones of the orthotic.

Still another aspect of the present invention is to provide sensors for the orthotic adapted to sense pressure and other biometrics.

Yet still another aspect of the present invention is to provide a customized orthotic including a bladder contacting the wound environment.

Another aspect of the present invention is to provide a customized cranial orthotic.

Still another aspect of the present invention is to provide collapsible shell allowing for easier transport of orthotic.

Yet still another aspect of the present invention is to provide an orthotic for use about the skull.

Still another aspect of the present invention is to provide an orthotic for use about a limb or other body part.

Another aspect of the present invention is to provide an orthotic for human or veterinary usage.

Still another aspect of the present invention is to utilize semiconductors to assist in control the temperatures of the wound environment.

Yet still another aspect of the present invention is to utilize a combination of tunnels and fans connected to the shell to circulate air about the wound environment.

A preferred embodiment of the current invention can be described as a cranial orthotic comprising: a) an exterior shell comprising: i) sections allowing selective zonal adjustment of pressures supplied to the sections and a wound environment of a skull, wherein the sections are collapsible when the cranial orthotic is not in use; ii) a removable vent; and iii) one or more ports extending through the exterior shell; the one or more ports adapted to receive foam; b) a bladder contacting an inward side of the exterior shell, wherein the bladder can receive foam; c) an array comprising sensors and a junction; the array contacting the inward side of the exterior shell or the bladder or a combination thereof, wherein the sensors sense zonal pressures associated with one or more sections of the shell; d) a plurality of semiconductors comprising thermal interfaces and ferromagnetic properties; the plurality of semiconductors connected to an inward side of the exterior shell or the bladder or a combination thereof, wherein relative to the positioning of each of the semiconductors about the wound environment and dependent on a direction of current flowing through the thermal interfaces, the current causes the semiconductor to heat or cool an area of the wound environment associated with the semiconductor's footprint; e) a detachable communications module, distinct from the shell, the bladder, the foam, the array and the junction, comprising a housing; the housing comprising: i) a first face to magnetically reciprocate with the junction; ii) an outward face comprising a touchscreen; and iii) a computer module comprising one or more of the following components: a microprocessor, a memory, a visual graphics unit, an audio unit, a transmitter or transceiver and a software for controlling the components, the cranial orthotic and displays by the touchscreen; f) the transmitter or transceiver adapted for wireless communications, via an available wireless cellular network and/or IEEE 802.11 protocol, with a cloud server or other computer remote from the communications module; and g) circuitry providing connections between the communications module, the array, the sensors and the semiconductors and a power source for the communications module.

Another preferred embodiment of the current invention can be described as an orthotic comprising: a) an exterior shell comprising one or more ports extending through the exterior shell; the one or more ports adapted to receive foam; b) an array comprising sensors and a junction; the array contacting a portion of and distinct from an inward side of the exterior shell, wherein the sensors sense zonal pressures associated with one or more sections of the shell; c) a detachable communications module comprising a housing; the housing, distinct from the shell, the foam, the array and the junction, comprising: i) a first face adapted to reciprocate with the junction; ii) a second face comprising a touchscreen; and iii) a computer module comprising one or more of the following components: a microprocessor, a memory, a visual graphics unit, an audio unit, a transmitter or transceiver and a software for controlling the components, the orthotic and displays by the touchscreen; d) the transmitter or transceiver adapted for wireless communications, via a wireless cellular network and/or IEEE 802.11 protocol, with a cloud server or other computer remote from the communications module; and e) circuitry providing connections between the communications module, the array, the sensors and a power source for the communications module.

Still another preferred embodiment of the current invention can be described as a detachable communications module adapted for use with a customizable orthotic helmet, cap or splint; the detachable communications module comprising a housing comprising: a) a first face adapted to reciprocate electromagnetically with a junction of an array conformed to fit about a wound environment facing side of a shell of the customizable orthotic helmet, cap or splint; the array comprising sensors and a junction; b) a computer module comprising one or more of the following components: a microprocessor, a memory, a visual graphics unit, an audio unit, a transmitter or transceiver and a software for controlling the components, the customizable orthotic and displays by the touchscreen; the transmitter or transceiver adapted for wireless communications, via a wireless cellular network and/or IEEE 802.11 protocol, with a cloud server or other computer remote from the communications module; c) circuitry providing connections between the communications module, the array, the sensors positioned on the shell of the orthotic helmet, cap or splint or a bladder inward from the shell and proximate the wound environment or both the shell and the bladder and a power source for the communications module; and d) a touchscreen positioned on a second side of the housing displaying data associated with wound environment to a user, thereby allowing the user of the communications module to control pressures supplied to the wound environment.

It is the novel and unique interaction of these simple elements which creates the system within the ambit of the present invention. Pursuant to Title 35 of the United States Code, select preferred embodiments of the current invention follow. However, it is to be understood that the descriptions of the preferred embodiments do not limit the scope of the present invention.

### BRIEF DESCRIPTION OF THE DRAWINGS

FIG. 1 is a perspective of cap or helmet (20).

FIG. 2 is a perspective of outer shell and pneumatic bladder of helmet (20).

FIG. 3 portrays a method of creating foam for helmet (20).

FIG. 4 is a perspective of cap or helmet (20).

FIG. 5 is a perspective of array (48) adapted for fitting into helmet (20).

FIG. 6 is a lateral view of cap or helmet (20).

FIG. 7 is perspective of the inside of cap or helmet (20).

FIG. 8 is perspective of the inside of cap or helmet (20).

FIG. 9 is a perspective of an upper portion of cap or helmet (20).

FIGS. 10-18 are view of embodiments of the current cap or helmet (20).

### DESCRIPTION OF THE PREFERRED EMBODIMENTS

Although the disclosure hereof is detailed to enable those skilled in the art to practice the invention, the embodiments published herein merely exemplify the present invention.

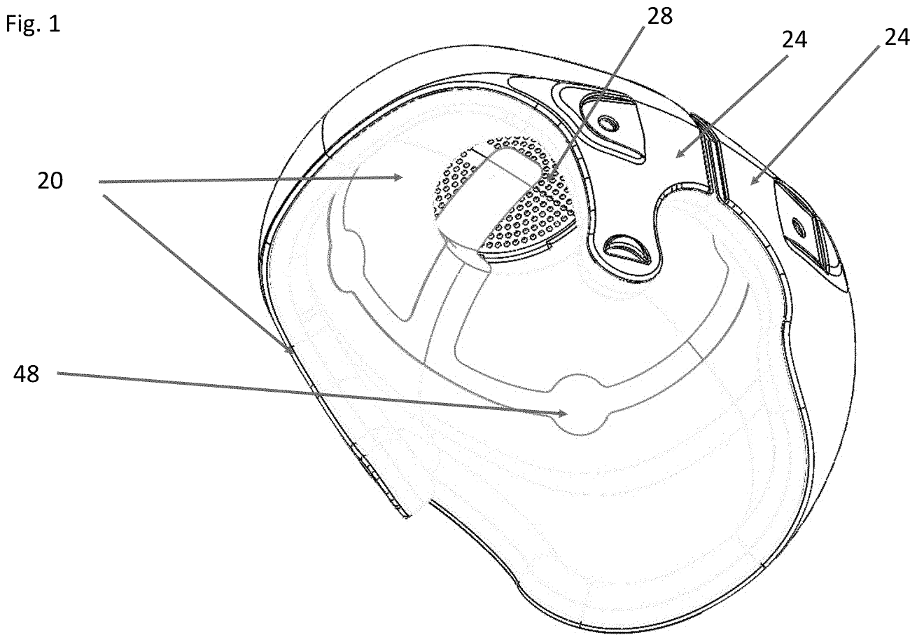

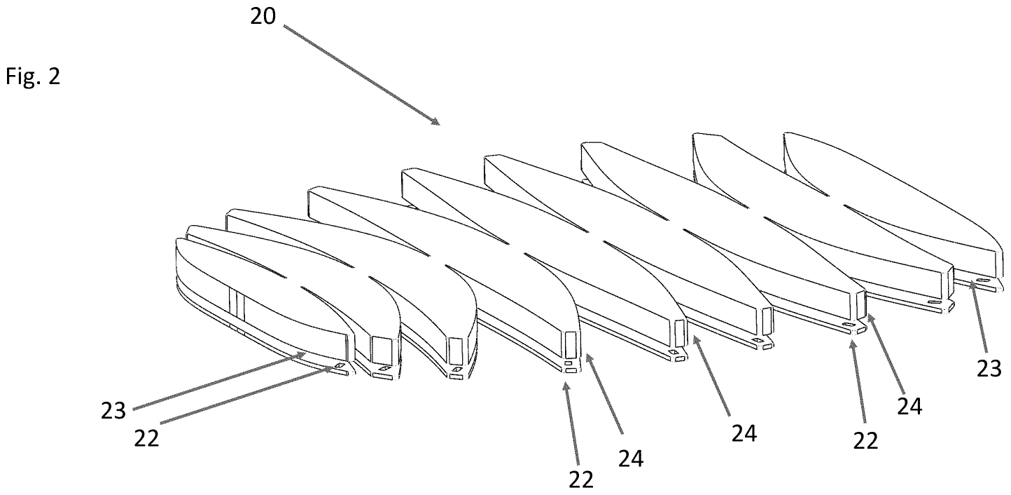

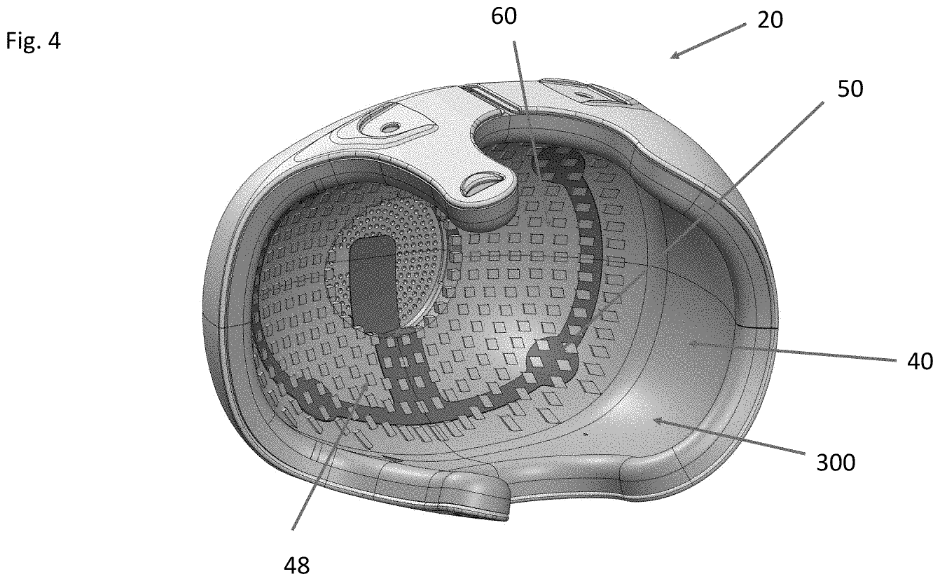

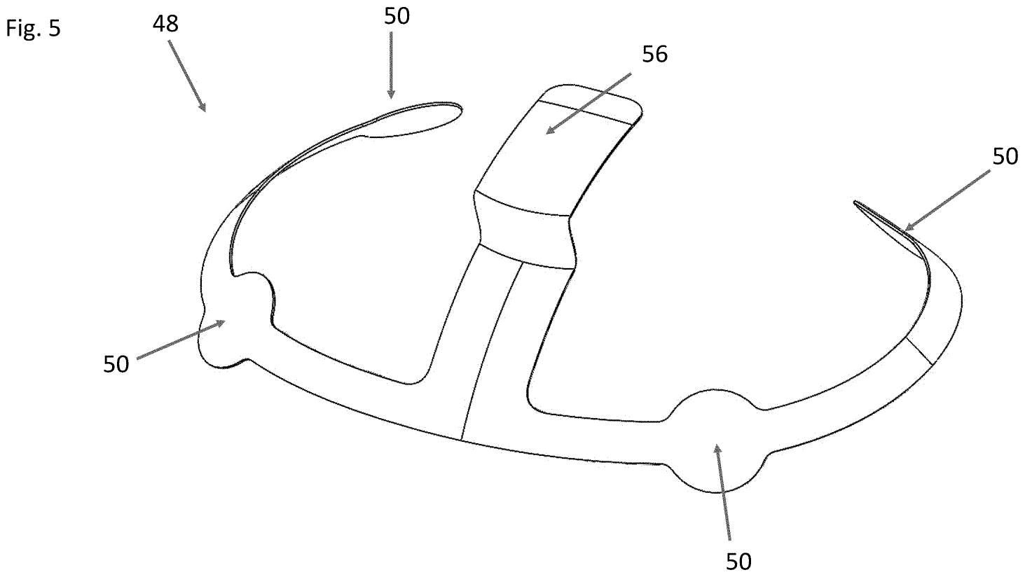

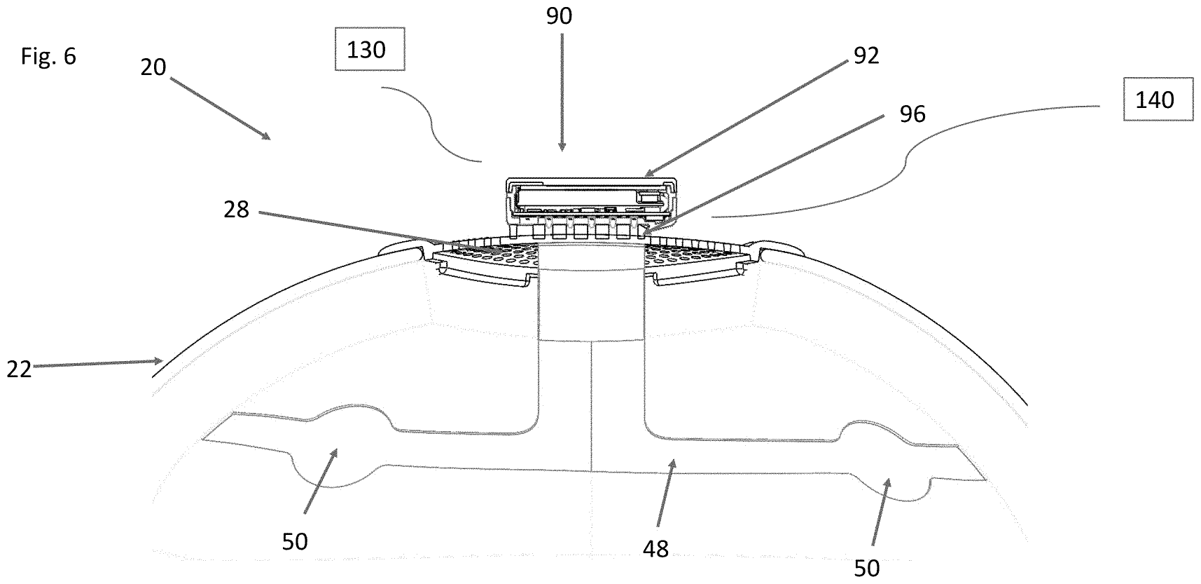

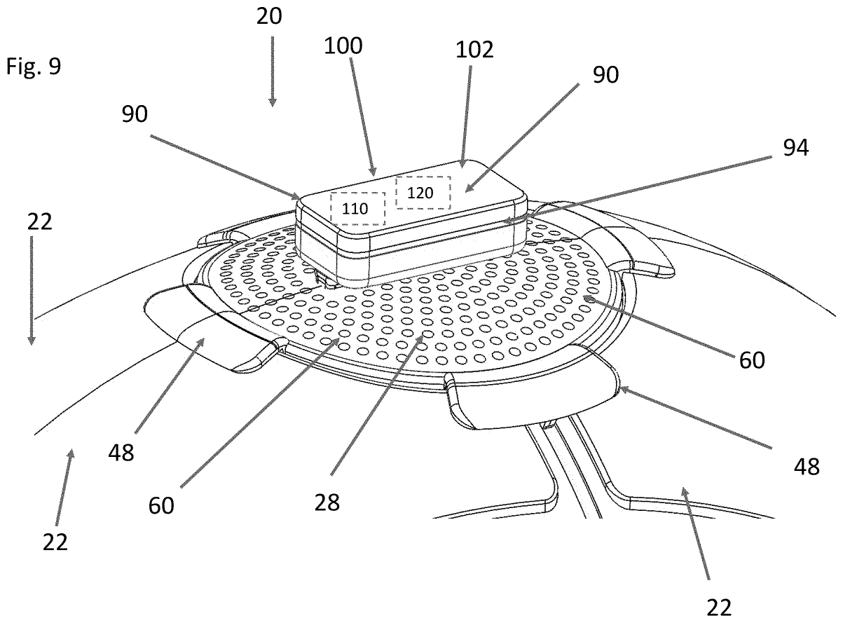

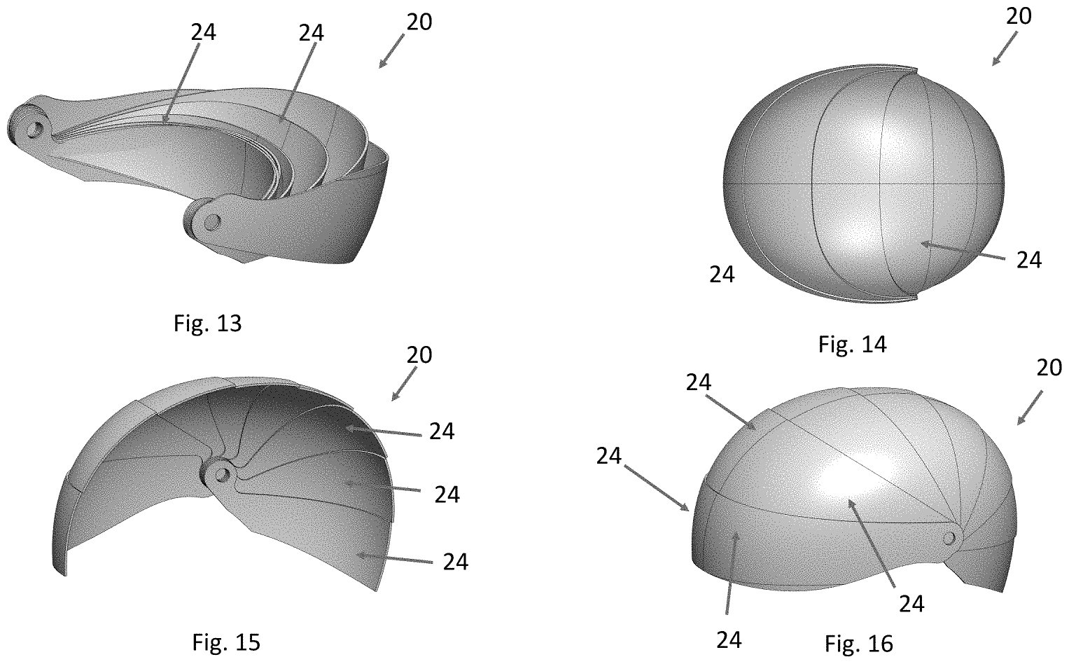

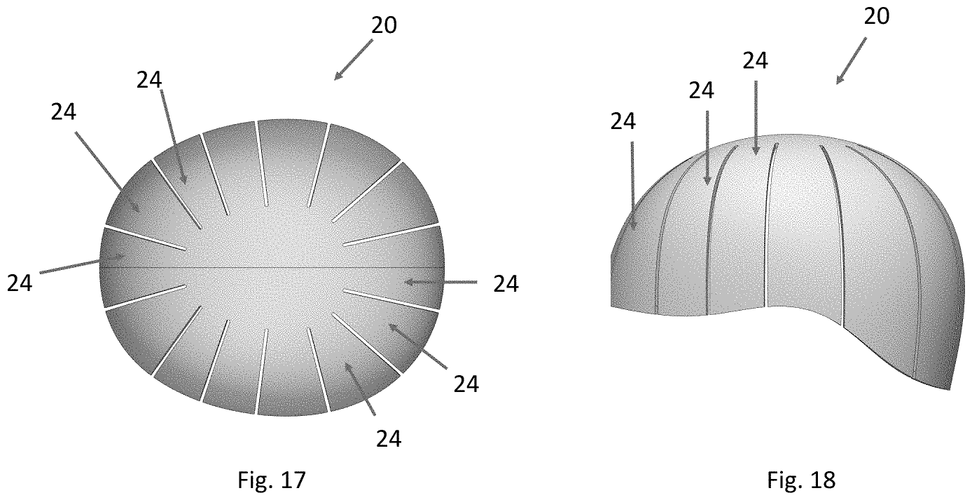

As described above and with reference to FIGS. 1-18, subsequent to a traumatic insult, the present smart custom cranial orthotic (20) can be formed to correspond to a soft tissue or bony defect in the skull or other bone.

FIG. 1 is a perspective of cap or helmet (20). By way of illustration, exterior shell (22) of helmet (20) can be constructed of polyurethane, carbon fiber, poly-para-phenylene terephthalamide (Kevlar), polycarbonate, polypropylene, injection molded plastic, high density polyethylene (HDPE), acrylonitrile butadiene styrene (ABS), other polymers and equivalent materials or combinations thereof. Sections or leaflets (24) of exterior shell (22) provide for selective zonal adjustment of pressures that can be supplied by adjustments of leaflets (24) to the wound environment (10). When not in use, sections (24) are collapsible allowing for easier transport of helmet or cap (20). In select preferred embodiments, helmet (20) is provided with removable vent (28) and chin strap (18). Removable vent (28) can improve air movement under helmet (20) to assist with heat control. Removal of vent allows better access to patient's scalp for probes, medication applications, visual observations, and when required, placement of one or more additional sensors without removing helmet (20) from the patient.

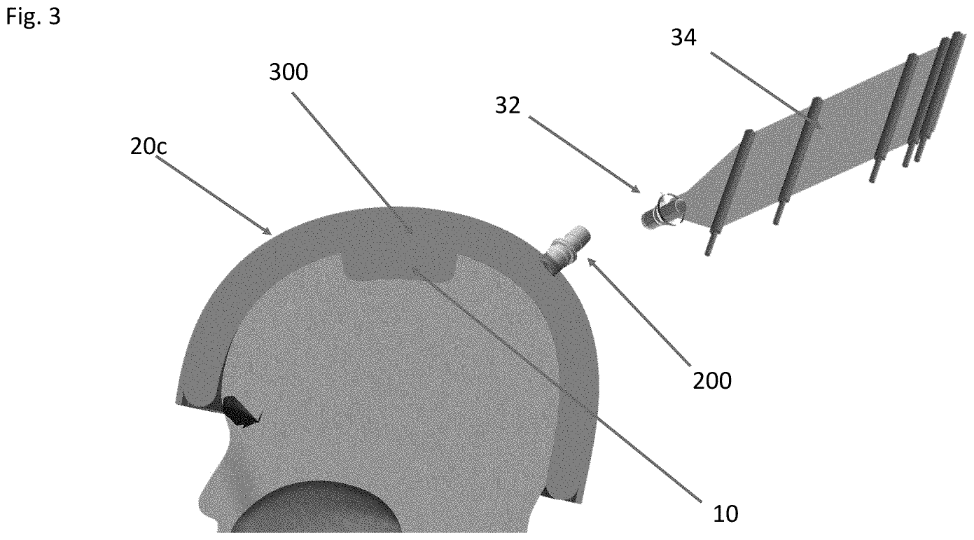

With a view toward FIG. 3, for select preferred embodiments of the current invention, via port (200) of helmet (20), a multipart liquid can be injected creating foam (300) that fills the volume of the wound environment. Depending on medical parameters of the wound environment (10), foam (300) can be customized for a patient. The created foam or foam layer (300) is positioned inward of exterior shell (22) of cap (20). External bag (34) via adapter (32) can be attached to port (200) of cap (20) to deliver an expanding liquid. For example, a 2-part silicone foam can be mixed in external packet (34). External packet (34) includes at least two compartmentalized sections. Once the seal between the sections is broken, a catalyst causes foaming of the expanding liquid and subsequent volume expansion. When external bag (34) is attached to the port (300), the contents of bag (34) can be extruded into foam layer (300) to fill an underlying tissue defect (10) in a closed environment. This use can provide custom compression on any morphology of the wound environment.

In use of helmet (20), bladder (40) contacts inward side (23) of exterior shell (22). When helmet (20) is provided with bladder (40), foam (300) can expand about a portion of bladder (40). Bladder (40) can function as an overflow valve for expanded foam (300), that when required can be trimmed and customized subsequent expansion of foam (300). Foaming agents may be composed of silicone, polyurethane, cellulose, bamboo, or other biodegradable agents and will be open cell of an open cell configuration to allow for application of negative pressure or evacuation of fluid when medically required. Foams (300) with open cell configurations allows for therapeutic agents to be applied to the wound environment (10) that can assist with hemostasis and healing. Reactions creating foams (300) biocompatible with tissues and are non-exothermic or gas producing. When medical parameters require, an aerosol spray or mixing-tip gun can be utilized to create foams (300) for wound environment (10). It has been discovered that the process creating foam (300) and fitting cap or helmet (20) in situ can be accomplished with a set up time under 10 minutes.

Select preferred embodiments of the current helmet (20) can be provided with an array (48) of sensors (50) and a junction (56) for releasably holding communications module (90). Array (48) can contact or be printed onto inward side (23) of exterior shell (22). Junction (56) includes circuitry allowing intercommunications with sensors (50) and communications module (90). Within the ambit of the current invention, junction (56) can be provided with a magnetic electroconductive T-pin connectable with communications module (90). When medical parameters require, the locations and number of sensors (50) positioned on array (48) can be altered and other sensors (50) can be incorporated into cap (20), bladder (40) or foam (300). Sensors (50) can sense biometrics such as pressure, temperature, blood pressure, lactate levels, pH, growth factors, hydrogen levels, sodium levels, potassium level, lactate levels, other electrolytes, cytokines, glucose levels, apoptotic factors, SVO2 and other biometric metric markers. Electroactive inks can be a sensor and printed directly or laminated through materials such as thermoplastic polyurethane (TPU) with an adhesive onto shell (22), bladder (40) or foam (300). Foams (300) can be composed of carbon nanotubules or multi-channel carbon-nanotubes to capture and sense specific ions, including but not limited to, H+, lactate, Na+, K+, glucose, cytokines, growth factors, other electrolytes or biometric markers.

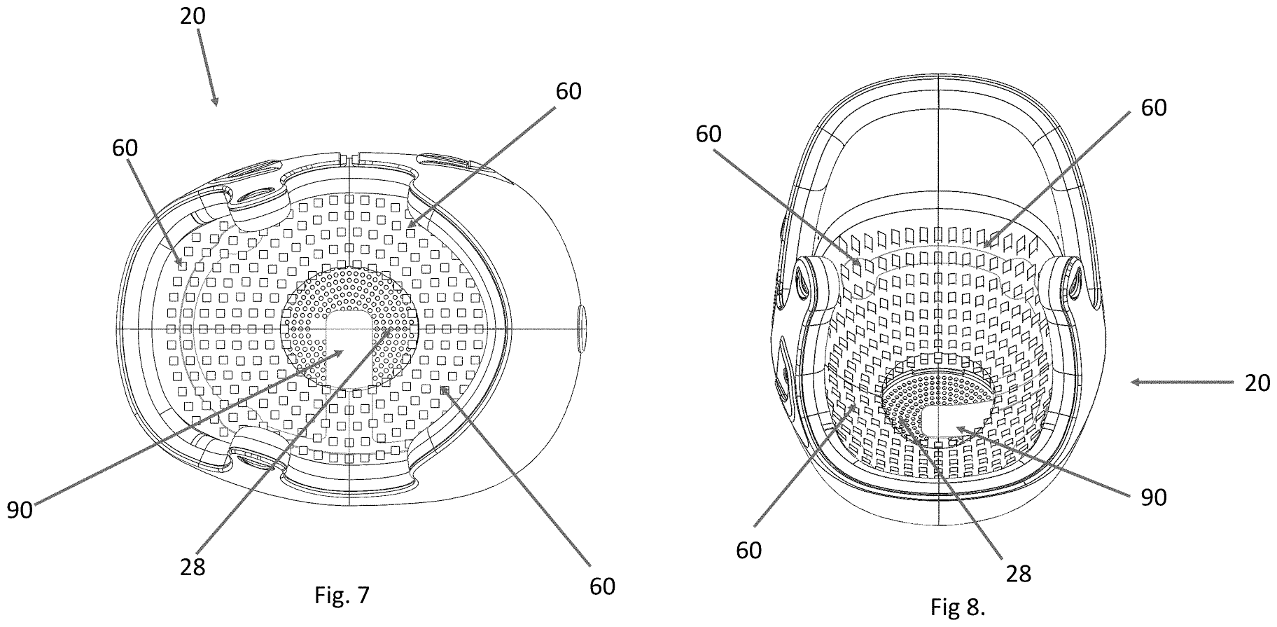

FIGS. 7-9 portray semiconductors (60), such micro-Peltier devices, connected to inner side (23) of exterior shell (22), bladder (40), foam (300) or any combination thereof. Semiconductors (60) can have a footprint of less than 2 millimeters² and the required circuitry interconnects each semiconductor (60) to junction (56) or another connector associated with cap (20). Heat/cool software controls the direction current flow for each semiconductor that results in semiconductor's (60) heating or cooling. Heat/cool software can be associated with memory of communications module (90) or at a location remote from helmet (20). Among other things, heat/cool software can generate a heat map of temperatures within helmet (30) to better quantify therapeutic intervention required. Whether in the field or the hospital, it is believed that the selective control of temperatures generated by semiconductors (60) inside helmet (20) can improve medical outcomes for the patient.

Communications module (90) includes housing (92). Alarm (94) can audible or visual such as a LED positioned on outward side of housing (92). Alarm (94) is activated when a sensed pressure or biometric for the patient is outside of a predetermined range. The LED light ring changes colors when the sensed pressure or biometric is outside the predetermined range. By way of illustration, LED (94) can turn red with the pressures in helmet (20) are calculated to be excessive or blue to designate helmet (20) is incorrectly fitted, positioned or at risk for movement.

Housing (92) is provided with first face (96) adapted to be received by junction (56). In select preferred embodiments, a magnetic attraction between first face (96) and junction (56) assist in securing electrical connections between first face (96) and junction (56). As previously indicated, junction (56) can include a magnetic electroconductive T-pin connect to first face (96). Housing (92) can be provided with a tongue, rail or other device to assist in attaching housing (92) to shell (22) of helmet (20).

Housing (92) can be provided with second or outward or second face (100) visible by the user of communications module (90). An OLED or AMOLED touch screen (102) can be incorporated into outward face (100) to provide user access to communications module's (90) computer module (110) can be provided with one or more of the following components: microprocessor, memory, visual graphics unit, audio unit, transmitter or transceiver (120), circuitry interconnecting the components and software (180) for controlling the components and cap (20). Among other things, computer component can calculate pressures and biometrics sensed by sensors (50), control current flow to semiconductors (60) and generate displays of data on touchscreen (102). Within the scope of the current invention, touchscreen (102) can display data correlated and sensed by sensors (50) and semiconductors (60). The visualized, calculated and correlated data can be supplied detachable communications module (90), cloud server (140) or computer remote (130) remote from communications module (90) or a combination thereof allowing the user of touchscreen (102) to control application of pressure to wound environment (20). Pressures supplied to wound environment (10) can also be controlled by cloud server (140) or computer remote (130) remote from communications module (90). Touchscreen (102) can also display correlated data from biometric sensors (50) sensing one or more of pressure, temperature, blood pressure, lactate levels, pH, growth factors, hydrogen levels, sodium levels, potassium level, lactate levels, other electrolytes, cytokines, glucose levels, apoptotic factors or SVO2.

Helmet (20) is adapted for wireless communications via any wireless network such as available cellular networks and/or IEEE 802.11 protocol at a frequency of 2.4 GHz (Wi-Fi or Bluetooth). Transmitter or transceiver (120) can communicate with a Cloud server or computer (140), other computer (130) such as a mobile computing device (smart phone, tablet, etc.) or a personal computer or any type of computing device remote from orthotic (20). Among other things, devices remote from helmet (20) can be utilized to store data, calculate biometrics and/or control pressures applied by helmet (20) to the wound environment (10). For select preferred embodiments of sensors (50), one or more sensors (50) can be equipped with wireless capabilities to communicate wirelessly with communications module (90), cloud server or other computer (130).

Power sources for communications module (90) include but are not limited to rechargeable sources such as lithium ion, lithium iron phosphate, solid state, tab-less batteries or other usable power sources. Within the scope of the invention, the power source can be attached to housing (92). The rechargeable power sources can be detachable from housing (92) to engage the recharging energy supply.

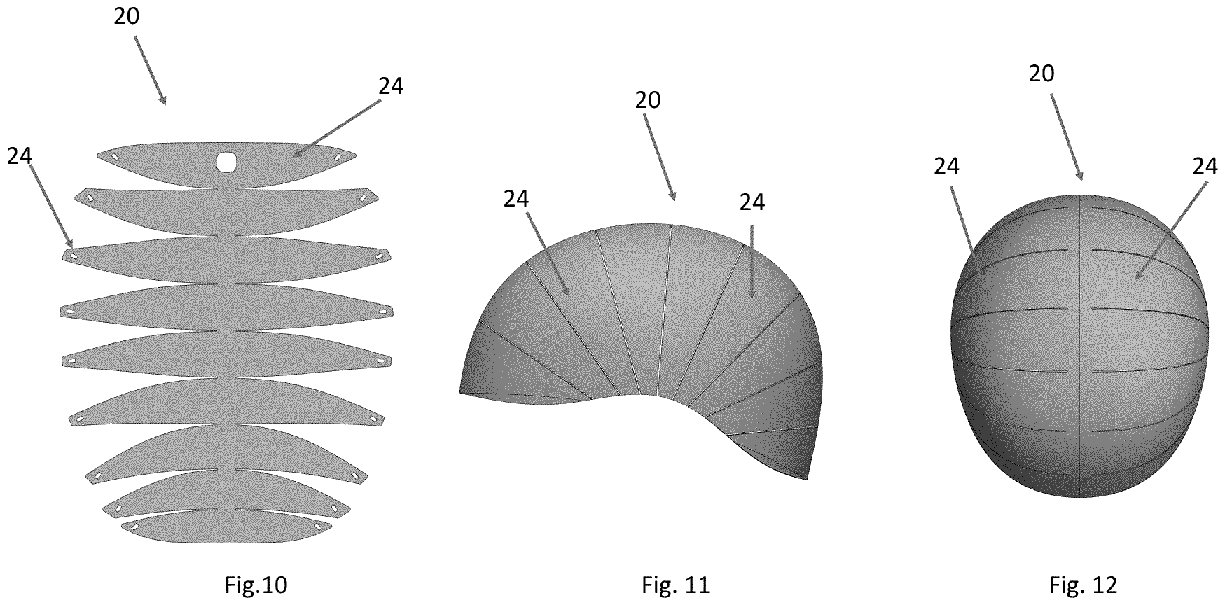

FIGS. 10-18 disclose different shells (22) of the current helmet (20). Sections or leaflets (24) are collapsible and can be flattened for more efficient storage. Among the many potential configurations for shells (22), gore map design, armadillo or nautilus telescoping segments and tulip flanges are portrayed. Although not shown, draw strings, zip ties, laces, hook and loop fasteners and Boa-type clip technology can assist with rapid formation of the shape of the in-use cap or helmet (20).

Select preferred embodiments of the current invention have been disclosed and enabled as required by Title 35 of the United States Code.

What is claimed is:

1) A cranial orthotic comprising:
a) an exterior shell comprising:
i) sections allowing selective zonal adjustment of pressures supplied to the sections and a wound environment of a skull, wherein the sections are collapsible when the cranial orthotic is not in use;
ii) a removable vent; and
iii) one or more ports extending through the exterior shell; the one or more ports adapted to receive foam;
b) a bladder contacting an inward side of the exterior shell, wherein the bladder can receive foam;
c) an array comprising sensors and a junction; the array contacting the inward side of the exterior shell or the bladder or a combination thereof, wherein the sensors sense zonal pressures associated with one or more sections of the shell;
d) a plurality of semiconductors comprising thermal interfaces and ferromagnetic properties; the plurality of semiconductors connected to an inward side of the exterior shell or the bladder or a combination thereof, wherein relative to the positioning of each of the semiconductors about the wound environment and dependent on a direction of current flowing through the thermal interfaces, the current causes the semiconductor to heat or cool an area of the wound environment associated with the semiconductor's footprint;
e) a detachable communications module, distinct from the shell, the bladder, the foam, the array and the junction, comprising a housing; the housing comprising:
i) a first face to magnetically reciprocate with the junction;
ii) an outward face comprising a touchscreen; and
iii) a computer module comprising one or more of the following components: a microprocessor, a memory, a visual graphics unit, an audio unit, a transmitter or transceiver and a software for controlling the components, the cranial orthotic and displays by the touchscreen;
f) the transmitter or transceiver adapted for wireless communications, via an available wireless cellular network and/or IEEE 802.11 protocol, with a cloud server or other computer remote from the communications module; and
g) circuitry providing connections between the communications module, the array, the sensors and the semiconductors and a power source for the communications module.

2) The cranial orthotic of claim 1 comprising biometric sensors sensing one or more of pressure, temperature, blood pressure, lactate levels, pH, growth factors, hydrogen levels, sodium levels, potassium level, lactate levels, other electrolytes, cytokines, glucose levels, apoptotic factors, nitric oxide levels or SVO2.

3) The cranial orthotic of claim 2 comprising an alarm, wherein the alarm is audible, visual or a combination thereof when the sensed pressure or other biometric for a patient is outside of a predetermined range.

4) The cranial orthotic of claim 1, wherein the display on the touchscreen was created by the communications module, the cloud server, the other computer remote from the communications module or a combination thereof.

5) The cranial orthotic of claim 4, wherein the display portrays a pressure map or a heat map or both associated with the wound environment.

6) The cranial orthotic of claim 4, wherein the thermal interfaces comprise carbon nanotubes adapted to decrease thermal interface resistance.

7) The cranial orthotic of claim 6, wherein one or more of the thermal interfaces induce vibration of fluids proximate the one or more of the thermal interfaces.

8) The cranial orthotic of claim 7 comprising tunnels and one or more fans connected to a surface of the cranial orthotic.

9) The cranial orthotic of claim 7, wherein up to about a 25 degree Celsius temperature differential is generated.

10) The cranial orthotic of claim 1, wherein the semiconductors are rigid or flexible or a combination thereof.

11) An orthotic comprising:
a) an exterior shell comprising one or more ports extending through the exterior shell; the one or more ports adapted to receive foam;
b) an array comprising sensors and a junction; the array contacting a portion of and distinct from an inward side of the exterior shell, wherein the sensors sense zonal pressures associated with one or more sections of the shell;
c) a detachable communications module comprising a housing; the housing, distinct from the shell, the foam, the array and the junction, comprising:
i) a first face adapted to reciprocate with the junction;
ii) a second face comprising a touchscreen; and
iii) a computer module comprising one or more of the following components: a microprocessor, a memory, a visual graphics unit, an audio unit, a transmitter or transceiver and a software for controlling the components, the orthotic and displays by the touchscreen;
d) the transmitter or transceiver adapted for wireless communications, via a wireless cellular network and/or IEEE 802.11 protocol, with a cloud server or other computer remote from the communications module; and
e) circuitry providing connections between the communications module, the array, the sensors and a power source for the communications module.

12) The orthotic of claim 7 comprising a bladder adapted to receive foam, wherein the bladder contacts the inward side of the exterior shell and the array contacts the inward side of the exterior shell or a portion of the bladder or both.

13) The orthotic of claim 12, wherein the first face reciprocates magnetically with the junction and the orthotic is a helmet, a cap or a splint.

14) The orthotic of claim 10 comprising a plurality of semiconductors comprising thermal interfaces; the plurality of semiconductors connected to inward side of the exterior shell or the bladder or a combination thereof, wherein relative to the positioning of each of the semiconductors about the wound environment and dependent on a direction of current flowing through the thermal interfaces, the current causes the semiconductor to heat or cool an area of the wound environment associated with the semiconductor's footprint.

15) The orthotic of claim 14 comprising:
a) sections allowing selective zonal adjustment of pressures supplied to the sections and a wound environment, wherein the sections are collapsible when the orthotic is not in use; and
b) a removable vent.

16) The orthotic of claim 14 comprising:
a) biometric sensors sensing one or more of pressure, temperature, blood pressure, lactate levels, pH, growth factors, hydrogen levels, sodium levels, potassium level, lactate levels, other electrolytes, cytokines, glucose levels, apoptotic factors, nitric oxide levels or SVO2; and
b) the touchscreen displaying a pressure map or a heat map or both associated with the wound environment.

17) The orthotic of claim 14, wherein the semiconductors are rigid or flexible or a combination thereof.

18) The orthotic of claim 17, wherein:
a) the semiconductors comprise ferromagnetic properties; and
b) the thermal interfaces are adapted to induce vibration of fluids proximate the one or more of the thermal interfaces and comprise carbon nanotubes adapted to decrease thermal interface resistance.

19) The orthotic of claim 18 comprising tunnels and one or more fans connected to a surface of the orthotic.

20) A detachable communications module adapted for use with a customizable orthotic helmet, cap or splint; the detachable communications module comprising a housing comprising:
a) a first face adapted to reciprocate electromagnetically with a junction of an array conformed to fit about a wound environment facing side of a shell of the customizable orthotic helmet, cap or splint; the array comprising sensors and a junction;
b) a computer module comprising one or more of the following components: a microprocessor, a memory, a visual graphics unit, an audio unit, a transmitter or transceiver and a software for controlling the components, the customizable orthotic and displays by the touchscreen; the transmitter or transceiver adapted for wireless communications, via a wireless cellular network and/or IEEE 802.11 protocol, with a cloud server or other computer remote from the communications module;
c) circuitry providing connections between the communications module, the array, the sensors positioned on the shell of the orthotic helmet, cap or splint or a bladder inward from the shell and proximate the wound environment or both the shell and the bladder and a power source for the communications module; and
d) a touchscreen positioned on a second side of the housing displaying data associated with wound environment to a user, thereby allowing the user of the communications module to control pressures supplied to the wound environment.

21) The detachable communications module of claim 20 communicating with and controlling one or more semiconductors positioned on the shell, the bladder or both; the semiconductors adapted to heat or cool an area of the wound environment.

22) The detachable communications module of claim 21, wherein the touchscreen displays:
a) data correlated and sensed by the sensors and received from the semiconductors; the correlated data supplied by the detachable communications module, the cloud server or the computer remote from the communications or a combination thereof allowing the user of the touchscreen to control application of pressure to the wound environment; the correlated data comprising biometric sensor data selected from the group consisting of one or more of the following: pressure, temperature, blood pressure, lactate levels, pH, growth factors, hydrogen levels, sodium levels, potassium level, lactate levels, other electrolytes, cytokines, glucose levels, apoptotic factors, nitric oxide levels or SVO2; and
b) a pressure map or a heat map or both associated with the wound environment.

23) The detachable communications module of claim 22, wherein the housing comprises an audible alarm, a visible alarm or both when the sensed pressure or other biometric for a patient is outside of a predetermined range.
